(19) United States
(12) Patent Application Publication
(10) Pub. No.: US 2009/0105488 A1
(43) Pub. Date: Apr. 23, 2009

(54) TRIPTYCENE DERIVATIVES AND THEIR APPLICATION

(75) Inventors: Chien-Hong Cheng, Hsinchu (TW); Hung-Hsin Shih, Hsinchu (TW); Ho-Hsiu Chou, Hsinchu (TW); Yu-Chen Jao, Hsinchu (TW)

Correspondence Address: WPAT, PC, 7225 BEVERLY ST., ANNANDALE, VA, 22003, US

(73) Assignee: Chien-Hong Cheng, Hsinchu (TW)

(21) Appl. No.: 11/876,454

(22) Filed: Oct. 22, 2007

(51) Int. Cl. C07D 413/14; C07C 211/61; C07C 43/21; C07D 413/02; C07F 7/08; C07F 9/28; H01J 1/63

(52) U.S. Cl. 548/440; 564/426; 568/633; 556/489; 568/14; 313/504

(57) ABSTRACT

The present invention discloses triptycene derivatives and their application as a host emitting material, an electron transport material, or a hole transport material in an organic electronic device. The triptycene derivative has the following general structure:

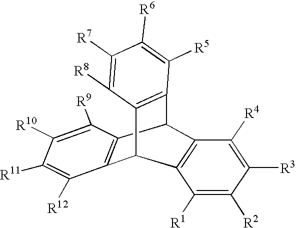

where R¹˜R¹² can be identical or different and R¹˜R¹² are independently selected from the group consisting of the following: aryl group having one or more substituents; heterocyclic aryl group having one or more substituents; non-aryl group having one or more substituents;

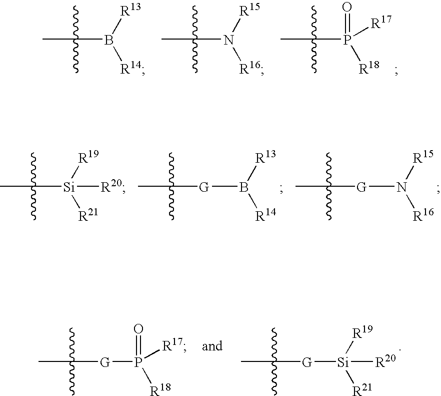

### REFERENCES CITED

Synthesis and Structure of 2,6,14- and 2,7,14-Trisunstituted Triptycene Derivatives., Chun Zhang; Chuan-Feng Chen, J. Org. Chem. 2006, 71, 6626-6629.

### BACKGROUND OF THE INVENTION

1. Field of the Invention

The present invention is generally related to a conjugated compound, and more particularly to triptycene derivatives and their application.

2. Description of the Prior Art

At present, phosphorescent metal complexes have been used as phosphorescent dopants in an organic light emitting diode. Among these metal complexes used in the light-emitting layer of the organic light emitting diode, cyclometalated iridium complexes have been extensively researched since their electron configurations have strong spin-orbit coupling. Since spin-orbit coupling results in mixing between the singlet and triplet excited states, the lifetime of the triplet state is greatly reduced and thereby the phosphorescence efficiency is promoted. In addition, it is found that the doping method can also enhance the efficiency of the device. Therefore, the method of doping phosphorescent substance in a host material is utilized and thus the research in blue phosphorescent host materials becomes important. In the earlier reports, the majority of the blue phosphorescent host materials are carbazoles. Carbazole derivatives have high triplet-state energy and are suitable as the blue phosphorescent host materials. In view of the above matter, developing a novel carbazole derivative having high triplet-state energy to prolong the usage lifetime of the device and to increase luminance efficiency is still an important task for the industry.

### SUMMARY OF THE INVENTION

In light of the above background, in order to fulfill the requirements of the industry, the present invention provides a novel triptycene derivative and its application as a host material, an electron transport material, or a hole transport material in an organic electronic device.

One object of the present invention is to provide a triptycene derivative having high heat stability to promote the usage lifetime of an organic electronic device.

Another object of the present invention is to provide a conjugated compound having diphenyl-silane structure having high triplet-state energy difference, which can not be provided by the general blue phosphorescence host materials, and can be used together with various common phosphorescent materials, such as blue, green, and red phosphorescent materials, like iridium (Ir), platinum (Pt), and osmium (Os) metal complexes. Therefore, this present invention does have the economic advantages for industrial applications.

Accordingly, the present invention discloses a triptycene derivative and its application as a host material, an electron transport material, or a hole transport material in an organic electronic device. The triptycene derivative has the following general structure:

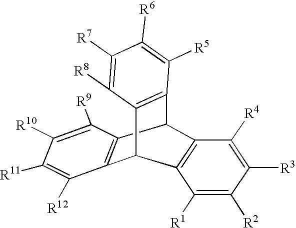

where R¹˜R¹² can be identical or different and R¹˜R¹² are independently selected from the group consisting of the following: aryl group having one or more substituents; heterocyclic aryl group having one or more substituents; non-aryl group having one or more substituents;

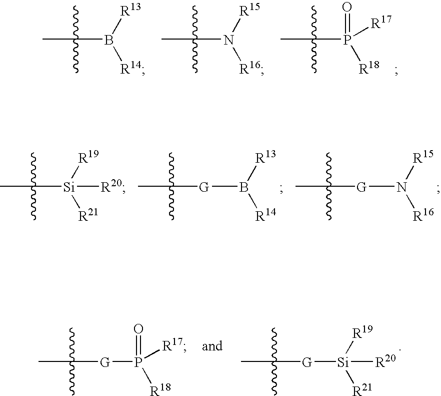

The above G is selected from the group consisting of the following: aryl group having one or more substituents; heterocyclic aryl group having one or more substituents; and heterocyclic group having one or more substituents. The invention also discloses the application of the triptycene derivative, especially the application as a host material, an electron transport material, a hole transport material, and an emitting host in an organic electroluminescence device or phosphorescence device; or the application as an electron transport material and a hole transport material in other organic electronic devices.

### DESCRIPTION OF THE PREFERRED EMBODIMENTS

What is probed into the invention is a triptycene derivative together with its application. Detail descriptions of the processes and composition structures will be provided in the following in order to make the invention thoroughly understood. Obviously, the application of the invention is not confined to specific details familiar to those who are skilled in the art. On the other hand, the common processes and composition structures that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In a first embodiment of the present invention, a triptycene derivative is disclosed. The triptycene derivative has the following general structure:

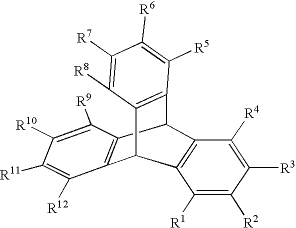

where R¹˜R¹² can be identical or different and R¹˜R¹² are independently selected from the group consisting of the following: aryl group having one or more substituents; heterocyclic aryl group having one or more substituents; non-aryl group having one or more substituents;

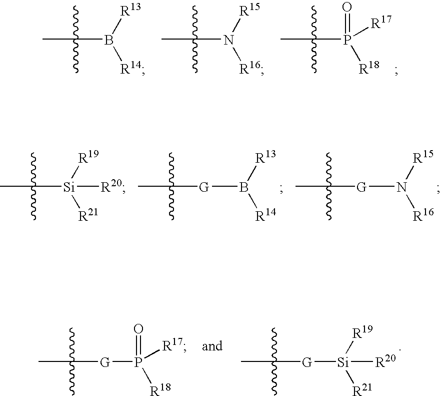

The substituent is selected from the group consisting of the following: H atom, halogen atom (such as F, Cl, Br, I), aryl group, halogen substituted aryl group, halogen substituted aralkyl group, haloalkyl substituted aryl group, haloalkyl substituted aralkyl group, aryl substituted C1-C20 alkyl group, electron donating group such as C1-C20 alkyl group, C1-C20 cycloalkyl group (such as methyl, ethyl, butyl, cyclohexyl), C1-C20 alkoxy group, C1-C20 substituted amino group, substituted aromatic amino group (such as aniline); or electron withdrawing group [such as halogen atom, nitrile group, nitro group, carbonyl group, cyano (—CN) group] and halogen substituted C1-C20 alkyl group (such as CF₃); and heterocyclic group.

The above G is selected from the group consisting of the following: aryl group having one or more substituents; heterocyclic aryl group having one or more substituents; heterocyclic group having one or more substituents. The substituent of G is selected from the group consisting of the following: H atom, halogen atom (such as F, Cl, Br, I), aryl group, halogen substituted aryl group, halogen substituted aralkyl group, haloalkyl substituted aryl group, haloalkyl substituted aralkyl group, aryl substituted C1-C20 alkyl group; electron donating group such as C1-C20 alkyl group, C1-C20 cycloalkyl group (such as methyl, ethyl, butyl, cyclohexyl), C1-C20 alkoxy group, C1-C20 substituted amino group, substituted aromatic amino group (such as aniline); or electron withdrawing group [such as halogen atom, nitrile group, nitro group, carbonyl group, cyano (—CN) group] and halogen substituted C1-C20 alkyl group (such as CF₃); and heterocyclic group.

The above R¹³˜R²¹ can be identical or different and R¹³˜R²¹ are independently selected from the group consisting of the following: H atom, C1-C20 alkyl group, C1-C20 cycloalkyl group (such as methyl, ethyl, butyl, cyclohexyl); C1-C20 alkoxy group; amino group; aryl group having one or more substituents; and heterocyclic aryl group having one or more substituents. The substituent of R¹³˜R²¹ is independently selected from the group consisting of the following: H atom, halogen atom, aryl group, halogen substituted aryl group, halogen substituted aralkyl group, haloalkyl substituted aryl group, haloalkyl substituted aralkyl group, aryl substituted C1-C20 alkyl group, C1-C20 alkyl group, C1-C20 cycloalkyl group, C1-C20 alkoxy group, C1-C20 substituted amino group, substituted aromatic amino group, electron withdrawing group substituted C1-C20 alkyl group, halogen substituted C1-C20 alkyl group (such as CF₃), heterocyclic group, nitrile group, nitro group, carbonyl group, and cyano group (—CN).

According to this embodiment, in the structure of the triptycene derivative, R¹˜R¹² are not H atoms simultaneously.

The aryl group is selected from the group consisting of the following: phenyl, naphthyl, diphenyl, anthryl, pyrenyl, phenanthryl, fluorene, or other multi-phenyl group.

The heterocyclic aryl group is selected from the group consisting of the following: pyrane, pyrroline, furan, benzofuran, thiophene, benzothiophene, pyridine, quinoline, isoquinoline, pyrazine, pyrimidine, pyrrole, pyrazole, imidazole, indole, thiazole, isothiazole, oxazole, isoxazole, benzothiazole, benzoxazole, 1,2,4-triazole, 1,2,3-triazole, 1,2,3,4-tetraazole, phenanthroline, or other heterocyclic aryl group.

The non-aryl group is selected from the group consisting of the following or any combination thereof: H atom, halogen atom (such as F, Cl, Br, I); C1-C20 alkyl group, C1-C20 cycloalkyl group (such as methyl, ethyl, butyl, cyclohexyl); C1-C20 alkoxy group; amino group; nitrile group; nitro group; carbonyl group; cyano group (—CN); halogen substituted C1-C20 alkyl group (such as CF₃); aryl substituted C1-C20 alkyl group; aryl substituted amino group; and C1-C20 alkyl substituted amino group.

The preferred examples of the structure and fabricating method for the triptycene derivative according to the invention are described in the following. However, the scope of the invention should be based on the claims, but is not restricted by the following examples.

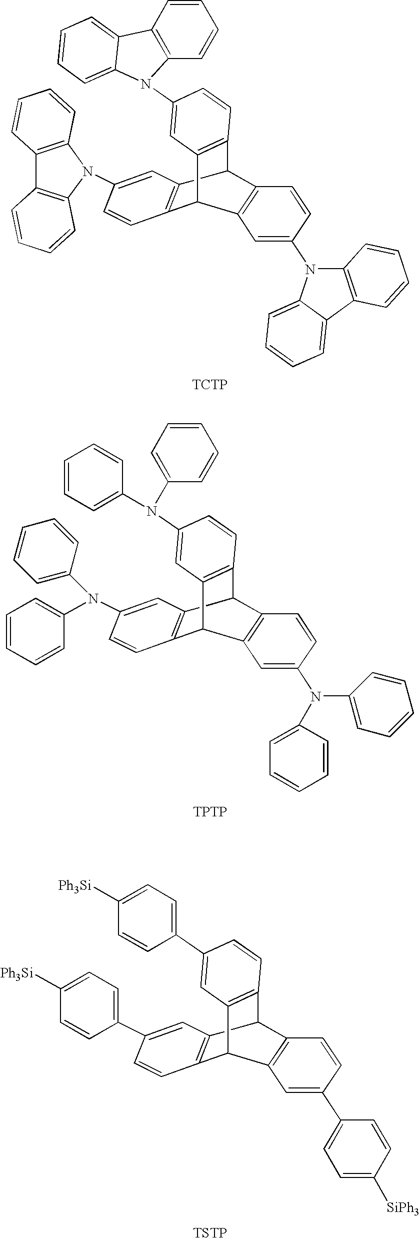

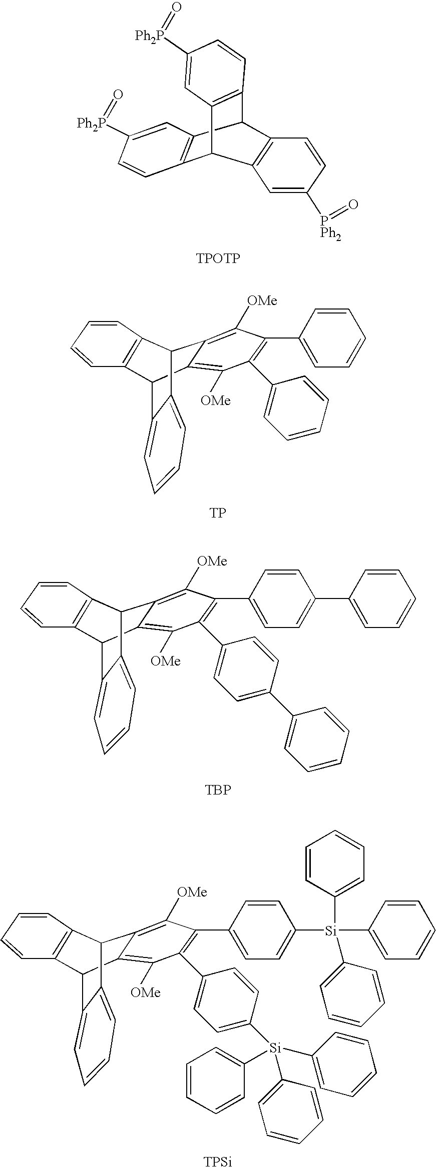

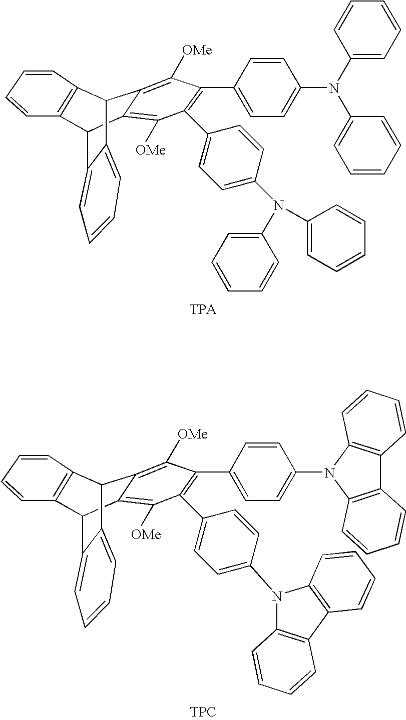

### EXAMPLE 1

### 2,6,14-tricarbazolyltriptycene (Hereinafter Abbreviated as TCTP)

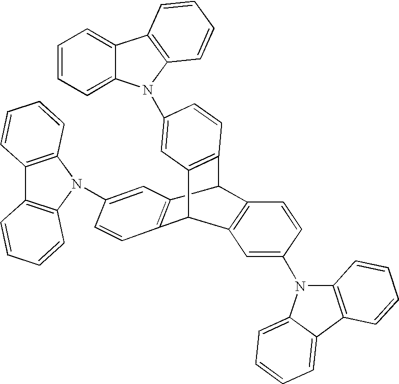

Carbazole (3.0 mmole, 0.5016 g) and the starting substance 2,6,14-triiodotriptycene (1.0 mmole, 0.632 g), and Pd(dba)₂ (0.06 mmole, 0.033 g) are taken and then placed in a high-pressure pipe. In a glove box, P(t-Bu)₃ [0.048 mmole, 0.096 g, 3 mL (10%, in Hexane)] and NaOtBu (4.5 mmole, 0.432 g) are added and 3 mL of xylene as a solvent is added. The pipe is then sealed in the glove box and is placed in a 150° C. silicone oil bath. The reaction is carried out for 72 hrs. After the reaction is finished, the mixture solution is stood for returning to room temperature. The solution is filtered by silica and tripoli and then washed by methylene chloride. The filtrate is collected and proper amount of active carbon is added. The filtrate is collected and dried to obtained yellowish solids. The yellowish solids are further washed by ether. White solids are collected, that is, the compound TCTP. The product yield is 50%.

¹H NMR (500 MHz, CDCl₃): δ: 8.14 (t, J=7.5 Hz, 6 H), 7.73-7.65 (m, 6 H), 7.46-7.37 (m, 12 H), 7.33-7.26 (m, 9 H), 5.71 (s, 1 H), 5.63 (s, 1 H). ¹³C NMR (125 MHz, CDCl₃): δ: 146.70, 146.57, 143.76, 143.62, 140.91, 140.88, 135.15, 135.11, 125.88, 125.19, 125.12, 124.22, 124.15, 123.31, 122.66, 122.62, 120.30, 119.96, 109.90, 109.86, 55.69, 53.48. HRMS (FAB⁺): Calcd for (C₅₆H₃₅N₃): 749.2831; found (M⁺) 749.2829.

### EXAMPLE 2

### 2,6,14-tris(diphenylamino)triptycene (Hereinafter Abbreviated as TPTP)

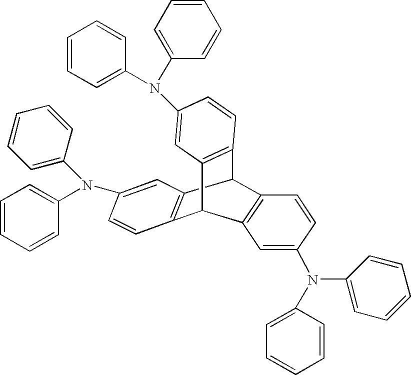

Bromobenzene (6.0 mmole, 0.942 g, 0.64 mL) and the starting substance 2,6,14-triaminotriptycene (1.0 mmole, 0.3 g), and Pd(dba)₂ (0.06 mmole, 0.033 g) are taken and then placed in a high-pressure pipe. In a glove box, P(t-Bu)₃ [0.048 mmole, 0.096 g, 3 mL (10%, in Hexane)] and NaOtBu (9.0 mmole, 0.864 g) are added and 3 mL of xylene as a solvent is added. The pipe is then sealed in the glove box and is placed in a 145° C. silicone oil bath. The reaction is carried out for 48 hrs. After the reaction is finished, the mixture solution is stood for returning to room temperature. The solution is filtered by silica and tripoli and then washed by methylene chloride. The filtrate is collected and dried to obtained yellowish solids. The yellowish solids are further washed by ether. White solids are collected, that is, the compound TPTP. The product yield is 76%.

¹H NMR(500 MHz, CDCl₃): δ: 7.20-7.16 (m, 12 H), 7.11-6.95 (m, 24 H), 6.68 (d, J=2 Hz, 1 H), 6.66 (d, J=2.5 Hz, 1 H), 6.64 (d, J=2 Hz, 1 H), 5.09 (s, 1 H), 4.98 (s, 1 H). ¹³C NMR (125 MHz, CDCl₃): δ: 147.91, 146,72, 144.94, 144.83, 139.77, 139.22, 129.14, 129.11, 124.31, 124.16, 124.06, 123.87, 122.52, 122.46, 120.30, 119.89, 119.63, 119.20, 53.37, 52.78. HRMS (EI⁺): Calcd for (C₅₆H₄₁N₃): 755.3300 found: (M⁺) 755.3300.

### EXAMPLE 3

### 2,5-dimethoxy-3,4-diphenyl-triptycene (Hereinafter Abbreviated as TP)

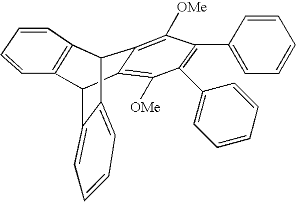

1-Phenyl boronic acid (12.3 mmole; 1.5 g) and 2,5-dimethoxy-3,4-dibromotriptycene (4.1 mmole; 1.9 g) are taken and then placed in a high-pressure pipe. Pd(PPh₃)₄ (5 mole %; 0.23 g) as the catalyt, 2M potassium carbonate solution (20 mmole; 2.8 g) as the base, and dimethoxyethane (DME) 8 mL as the solvent are added. Carbon-carbon bond addition Suzuki coupling reaction is then carried out. After the reaction is finished, methylene chloride is used as the elute and the organic layer solution quickly passes in the column chromatography. Then a clear yellow solution is separated and obtained. Anhydrous magnesium sulfate is used to remove water content. The organic solvent in the solution is dried by a rotary evaporator. Ether is added to wash and then white solids are obtained, that is, the compound TP. The product yield is 67%.

¹H NMR (400 M Hz, CDCl₃): δ 3.38 s, 6 H), 5.86 (s, 2 H), 6.93-6.96 (m, 4 H), 7.04-7.09 (m, 8 H), 7.49 (dd, J=5.2 Hz, J=3.2 Hz), 7.54 (d, J=7.2 Hz), 7.47 (dd, J=2 Hz, 4 H). ¹³C NMR (100 M Hz, CDCl₃): δ 48.5 CH), 61.4 (CH₃), 123.9 (CH), 125.4 (CH), 126.2 (CH), 127.2 (CH), 130.9 (CH), 133.2 (C), 136.6 (C), 138.4 (C), 145.3 (C), 149.0 (C). MS (EI, m/z): 466.1941 (M⁺). Anal. Calcd. for C₃₄H₂₆O₂: C, 87.28%; H, 5.54%. Found: C, 87.52%; H, 6.62%.

### EXAMPLE 4

### 2,5-dimethoxy-3,4-di-biphenyl-triptycene (Hereinafter Abbreviated as TBP)

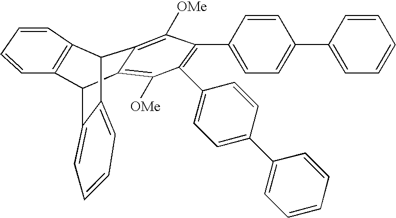

The synthetic method of TBP is the same as that in Example 3 except that the starting substance biphenyl-4-boronic acid is used instead of 1-phenyl boronic acid. Under the same reaction conditions, carbon-carbon bond coupling reaction is carried out. Due to different steric effect, the reaction time can be as long as 3-5 days. Finally, methanol is used to wash to obtain TBP solids. The product yield is 58%.

¹H NMR (400 MHz, CDCl₃): δ 3.43 (s, 6 H), 5.89 (s, 2 H), 7.06-7.08 (m, 8 H), 7.27 (t, J=7.2 Hz, 2 H), 7.35-7.38 (m, 8 H), 7.49 (dd, J=5.2 Hz, J=3.2Hz), 7.54 (d, J=7.2 Hz). ¹³C NMR (100 M Hz, CDCl₃): δ 48.6 (CH), 61.5 (CH₃), 123.9 (CH), 125.4 (CH), 125.9 (CH), 126.8 (CH), 127.1 (CH), 128.6 (CH), 131.4 (CH), 132.7 (C), 135.6 (C), 138.6 (C), 138.7 (C), 140.7 (C), 145.3 (C), 149.2 (C). Anal. Calcd. for C₄₆H₃₄O₂: C, 89.21%; H, 5.54%. Found: C, 89.29%; H, 5.54%.

### EXAMPLE 5

### 2, 5-dimethoxy-3,4-di-(4-triphenylsilyl-1-phenyl)-triptycene (Hereinafter Abbreviated as TPSi)

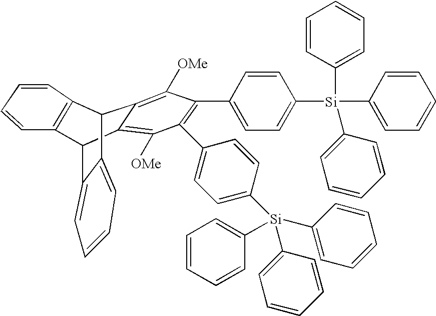

The synthetic method of TPSi is the same as that in Example 3 except that the starting substance 4-triphenylsilyl-1-phenylboronic acid is used instead of 1-phenyl boronic acid. Under the same reaction conditions, carbon-carbon bond coupling reaction is carried out. Due to different steric effect, the reaction time can be as long as 3-5 days. Finally, methanol is used to wash to obtain TPSi solids. The product yield is 60%.

¹H NMR (400 MHz, CDCl₃): δ 3.40 (s, 6 H), 5.86 (s, 2 H). 6.97 (d, J=8.4 Hz, 4 H), 7.04 (dd, J=5.2 Hz, J=3.2 Hz, 8 H), 7.19-7.25 (m, 12 H), 7.29-7.36 (m, 10 H), 7.43-7.47 (m, 16 H). ¹³C NMR (100 M Hz, CDCl₃): δ 48.5 (CH), 61.5 (CH₃), 123.9 (CH), 125.4 (CH), 127.8 (CH), 129.5 (CH), 130.5(CH), 131.7 (C), 132.8 (C), 134.2 (C), 135.2 (CH), 136.3 (CH), 137.9 (C), 138.6 (C), 145.2 (C), 149.0 (C). HRMS (FAB, m/z): calcd for C₅₀H₂₈F₂₄ 982.3662, found 983.3732 (M+H⁺). Anal. Calcd. for C₇₀H₅₄O₂: C, 85.09%; H, 5.39%. Found: C, 85.09%; H, 5.54%.

### EXAMPLE 6

### 2,5-dimethoxy-3,4-di-[4-(N,N-diphenylamino)-1-phenyl]-triptycene (Hereinafter Abbreviated as TPA)

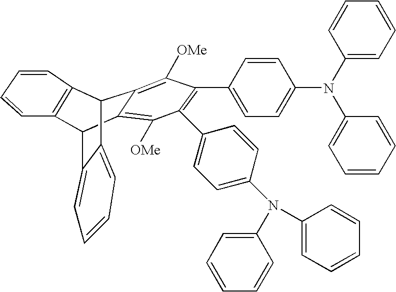

The synthetic method of TPA is the same as that in Example 3 except that the starting substance 4-(N,N-diphenylamino)-1-phenylboronic acid is used instead of 1-phenyl boronic acid. Under the same reaction conditions, carbon-carbon bond coupling reaction is carried out. Due to different steric effect, the reaction time can be as long as 3-5 days. Finally, methanol is used to wash to obtain TPA solids. The product yield is 65%.

¹H NMR (400 M Hz, CDCl₃): δ 3.49 (s, 6 H), 5.86 (s, 2 H), 6.81-6.87 (m, 8 H), 6.95 (t, J=7.2 Hz, 4 H), 7.00-7.05 (m, 8 H), 7.18 (t, J=7.2 Hz), 7.46 (dd, J=5.2 Hz, J=3.2 Hz). ¹³C NMR (100 M Hz, CDCl₃): δ 48.5 (CH), 61.4 (CH₃), 122.5 (CH), 122.8 (CH), 123.8 (CH), 124.1 (CH), 125.3 (CH), 129.2 (CH), 131.1 (C), 131.8 (CH), 132.9 (C), 138.2 (C), 145.3 (C), 145.8 (C), 147.7 (C), 149.1 (C). Anal. Calcd. for C₅₈H₄₄N₂O₂: C, 86.90%; H, 5.52%; N, 3.20%. Found: C, 86.97%; H, 5.54% N, 3.50%.

### EXAMPLE 7

### 2,5-dimethoxy-3,4-di[4-(9H-carbazol-9-yl) phenyl]-triptycene (Hereinafter Abbreviated as TPC)

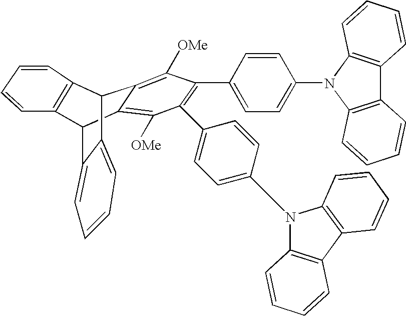

The synthetic method of TPC is the same as that in Example 3 except that the starting substance 4-(9H-carbazol-9-yl)phenylboronic acid is used instead of 1-phenyl boronic acid. Under the same reaction conditions, carbon-carbon bond coupling reaction is carried out. Due to different steric effect, the reaction time can be as long as 3-5 days. Finally, methanol is used to wash to obtain TPC solids. The product yield is 63%.

¹H NMR (400 MHz, CDCl₃): δ 3.60 (s, 6 H), 5.96 (s, 2 H), 7.11 (dd, J=5.6 Hz, J=3.2 Hz, 4 H), 7.21-7.23 (m, 8 H), 7.26-7.28 (m, 8 H), 7.39 (d, J=8 Hz, 4 H), 7.54 (dd, J=5.6 Hz, J=3.2 Hz), 8.09-8.11 (m, 4 H). ¹³C NMR (100 M Hz, CDCl₃): δ 48.6 (CH), 61.7 (CH₃), 109.5 (CH), 119.8 (CH), 120.2 (CH), 123.3 (C), 124.0 (CH), 125.5 (CH), 125.9 (CH), 126.0 (CH), 132.4 (CH), 132.5 (C), 135.9 (C), 136.0 (C), 139.2 (C), 140.7 (C), 145.1 (C), 149.1 (C).

### EXAMPLE 8

### 2,6,14-tris[4-(triphenylsilyl)phenyl]triptycene (Hereinafter Abbreviated as TSTP)

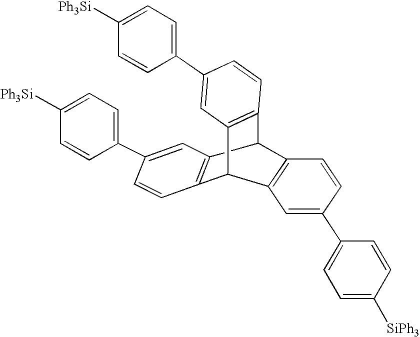

2,6,14-Triiodotriptycene (0.25 mmole, 0.1580 g), Pd(PPh₃)₄ (0.0375 mmole, 0.043 g), and 4-(triphenylsilyl)phenylboronic acid (1.25 mmole, 0.4754 g) are taken as the starting substances and then placed in a high-pressure pipe. K₂CO₃ solution (1 mmole, 0.138 g K₂CO₃ dissolved in 0.5 mL H₂O) is injected and 1,2-dimthoxyethane (1 mL) as the solvent is added. The pipe is then sealed and is placed in a 95° C. silicone oil bath. The reaction is carried out for 5 days. After the reaction is finished, the mixture solution is stood for returning to room temperature. The solution is filtered by silica and tripoli and then washed by methylene chloride. The filtrate is collected and column chromatography is used to perform purification. The compound TSTP is obtained. The product yield is 65%.

¹H NMR (400 MHz, CDCl₃): δ 5.56 (d, J=4.8 Hz, 2H), 7.23-7.25 (m, 3H), 7.34-7.44 (m, 30H), 7.49 (t, J=7.2 Hz, 9H), 7.55-7.57 (m, 21H), 7.65 (m, 3H).

### EXAMPLE 9

### 2,6,14-tris(diphenylphosphine oxide)triptycene (Hereinafter Abbreviated as TPOTP)

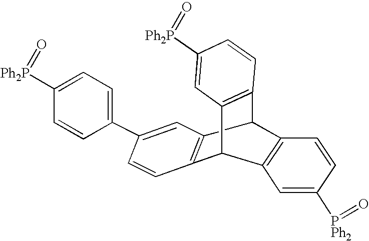

2,6,14-Triiodotriptycene (0.5 mmole, 0.3160 g) and Pd(OAc)₂ (0.003 mmole, 0.010 g) are taken as the starting substances and then placed in a high-pressure pipe. In a glove box, HPPh₂ (2.0 mmole, 0.3724 g) is added. The solution is taken out from the glove box and triethyl amine (NEt₃) and 2 mL of acetonitrile as a solvent are injected. The pipe is then sealed in the glove box and is placed in a 85° C. silicone oil bath. The reaction is carried out for 72 hrs. After the reaction is finished, the mixture solution is stood for returning to room temperature. The solution is filtered by silica and tripoli and then washed by methylene chloride. The filtrate is collected and column chromatography is used to perform purification. 2,6,14-Tris(diphenylphosphine)triptycene is thus obtained. 2,6,14-Tris(diphenylphosphine)triptycene is then dissolved in methylene chloride and 30% H₂O₂/H₂O is added therein. After stirred at room temperature for 24 hrs, the solution is extracted several times by methylene chloride. Anhydrous magnesium sulfate is used to remove water content. After filtration, the filtrate is collected and dried to obtained TPOTP. The product yield is 70%.

¹H NMR (400 MHz, CDCl₃): δ 5.48 (d, J=21.6 Hz, 2H), 7.14 (dd, J=12.4 Hz, J=7.2 Hz, 2H), 7.19-7.26 (m, 3H), 7.36 (dd, J=7.6 Hz, J=2.4 Hz, 2H), 7.4-7.6 (m, 12H), 7.51-7.54 (m, 5H), 7.59-7.65 (m, 12H), 7.72 (d, J=11.2 Hz, 3H), 7.82 (d, J=10.8 Hz, 1H).

### EXAMPLE 10

The major physical properties of the triptycene derivatives disclosed in Example 1 and Example 2 are measured and summarized in Table 1-1. And, the major physical properties of the triptycene derivatives disclosed in Example 8 and Example 9 are measured and summarized in Table 1-2.

TABLE 1-1

| | TCTP | TPTP |
|---|---|---|
| λ^{a}_{abs} (nm) | 343, 329, 294, 242 | 310, 227 |
| λ^{b}ₘₐₓ (nm) | 410, 438, 460 | 422, 438 |
| λ^{c}ₘₐₓ (nm) | 352 | 368 |
| triplet state energy^{d} (eV) | 3.02 | 2.94 |
| HOMO^{e} (eV) | 6.02 (5.86) | 5.66 (5.57) |
| LUMO (eV) | 2.40 (2.24) | 2.10 (2.01) |
| Tm^{f} (° C.) | 378.1 | 300.3 |
| Tg^{g}(° C.) | 237.8 | 178.4 |
| Tc^{h} (° C.) | xxx | 265.5 |

^{a}In UV-vis absorption measurement, CH₂Cl₂ is the solvent and the solution concentration is about 1 × 10⁻⁵ M;
^{b}Photoluminescence is measured at 77K by using EtOH as the solvent;
^{c}Photoluminescence is measured by using CH₂Cl₂ as the solvent and having solution concentration of about 1 × 10⁻⁵ M;
^{d}EtOH is used as the solvent and the measurement is carried out at 77 K;
^{e}Redox measurement is carried out in CH₂Cl₂ with solution concentration of about 1 × 10⁻³ M and the reported value is the value corresponding to Cp₂Fe/Cp₂Fe⁺;
^{f}Melting point
^{g}Glass transition temperature
^{h}Crystal-growth temperature

TABLE 1-2

| | TPOTP | TSTP |
|---|---|---|
| λ^{a}_{abs} (nm) | 272, 284 | 302 |
| λ^{b}ₘₐₓ (nm) | 390 | 482 |
| λ^{c}ₘₐₓ (nm) | 302 | 337 |
| triplet state energy^{d} (eV) | 3.18 | 2.72 |
| HOMO^{e} (eV) | 5.58 (5.52) | XXX |
| LUMO (eV) | 1.36 (1.30) | XXX |
| Tm^{f} (° C.) | 385.72 | XXX |
| Tg^{g}(° C.) | 190.17 | 220.13 |
| Tc^{h} (° C.) | 286.56 | 335.98 |

^{a}In UV-vis absorption measurement, CH₂Cl₂ is the solvent and the solution concentration is about 1 × 10⁻⁵ M;
^{b}Photoluminescence is measured at 77K by using EtOH as the solvent;
^{c}Photoluminescence is measured by using CH₂Cl₂ as the solvent and having solution concentration of about 1 × 10⁻⁵ M;
^{d}EtOH is used as the solvent and the measurement is carried out at 77 K;
^{e}Redox measurement is carried out in CH₂Cl₂ with solution concentration of about 1 × 10⁻³ M and the reported value is the value corresponding to Cp₂Fe/Cp₂Fe⁺;
^{f}Melting point;
^{g}Glass transition temperature;
^{h}Crystal-growth temperature.

### EXAMPLE 11

The major physical properties of the triptycene derivatives disclosed in Example 3Example 7 are measured and summarized in Table 2-1˜Table 2-2, where Table 2-1 shows the optical physical properties of the triptycene derivatives TP, TBP, TPA, TPSi, TPC and Table 2-2 shows the thermal properties of the triptycene derivatives TP, TBP, TPA, TPSi, TPC.

TABLE 2-1

| | λmax | λmax | λmax | λmax | | | |
|---|---|---|---|---|---|---|---|
| | Abs · in | EM in | EM | EM | | | |
| | DCM | DCM | (thin film) | (77K) | HOMO | E_{S} | E_{T}. |
| | (nm)^{a} | (nm)^{b} | (nm)^{c} | (nm)^{d} | (eV)^{e} | (eV) | (eV) |
| TP | 273 | 354 | 350 | 430 | — | 4.05 | 2.88 |
| TBP | 275 | 386 | 378 | 474 | 6.17 | 3.87 | 2.61 |
| TPA | 228; 310 | 400 | 392 | 450 | 5.51 | 3.48 | 2.75 |
| TPSi | 228; 242 | 364; 420 | 352; 382; | 442 | — | 4.05 | 2.80 |
| | | | 404 | | | | |
| TPC | 237; 294 | 352; 364 | 354; 398; | 412 | 6.13 | 3.62 | 3.01 |
| | | | 418 | | | | |

^{a}The solution concentration in UV-vis absorption measurement is about 1 × 10⁻⁵ M;
^{b}The solution concentration in photoluminescence measurement is about 1 × 10⁻⁵ M;
^{c}Thickness is about 300 nm;
^{d}It is measured in 2-methyl THF;
^{e}The measurement of HOMO uses ACII for TBP and CV for TPA and TPC;

TABLE 2-2

| | T_{g} (° C.)^{a} | T_{c} (° C.)^{b} | Tₘ (° C.)^{c} |
|---|---|---|---|
| TP | 113.4 | — | 248.4 |
| TBP | 149.1 | — | 286.5 |
| TPA | 143.4 | — | 340.3 |
| TPSi | 171.1 | 275.1 | 315.2 |
| TPC | 184.6 | — | 353.7 |

^{a}Heating rate and cooling rate are 10° C./min;
^{b}Crystal-growth temperature;
^{c}Heating rate and cooling rate are 20° C./min.

According to this embodiment, the triptycene derivative has excellent heat stability and high triplet-state energy difference. Therefore, as the triptycene derivative is applied in an organic electronic device, the excellent heat stability makes the lifetime of the organic electronic device increased. In addition, as the triptycene derivative is applied in an organic electroluminescence device, the triptycene derivative has high triplet-state energy difference, which can not be provided by the general host materials, and can be used together with various common emitting materials. For example, by doped with blue, green, and red phosphorescent materials, like iridium (Ir), platinum (Pt), and osmium (Os) metal complexes, the wavelength irradiated from the emitter layer can be adjusted according to actual needs.

In this embodiment, the triptycene derivative can be applied in an organic electroluminescence and/or phosphorescence device, especially used as a host material, an electron transport material, or a hole transport material. The triptycene derivative can also be applied as an electron transport material and a hole transport material in other organic electronic device. The organic electronic device can be a solar cell, an organic thin film transistor, an organic photoconductor, or other organic semiconducting device well-known to those who are skilled in the art.

In a second embodiment of the invention, an organic light emitting device is disclosed. Generally, the color of light emitted by the organic light emitting device is determined by the fluorescent organic material in the device. Therefore, by doping small amount of guest emitters with high luminance efficiency in host emtters, the recombination efficiency of carriers is increased. These guest emitters have smaller energy gap, higher luminance efficiency and shorter recombination lifetime than the host emitters. Therefore, the excitons of the host emitters quickly transfer to the guest emitters through energy transition to carry out recombination effectively. Besides increasing luminance efficiency, the color of the emitted light covers the whole visible light region.

Generally, guest emitters are used together with host emitters by co-evaporation or dispersion, or by spin coating. Guest emitters receive energy from the excited host emitters through energy transfer or carrier trap to produce different colors, such as red, green, and blue, and to increase luminance efficiency. Besides the above mentioned fluorescence guest emitters, new development in phosphorescence material is also researched. As an organic molecule is excited, one quarter of excited electrons form asymmetric spin siglet state and release energy through fluorescence. However, three quarters of excited electrons form symmetric spin triplet state but do not release energy through radiated phosphorescence to thereby lose efficiency. At present, the material capable of releasing the triplet-state energy of the excited electrons through radiated phosphorescence usually is an organic metallic compound having a center transition metal, such as osmium (Os), iridium (Ir), platium (Pt), europium (Eu), ruthenium (Ru), etc., and a nitrogen-containing heterocyclic compound as its ligand.

According to this embodiment, the organic light emitting device comprises a pair of electrodes and at least one organic layer provided between the electrodes. The at least one organic layer comprises one emitter layer and at least one of the organic layers comprises one compound containing a triptycene derivative, having the following general structure:

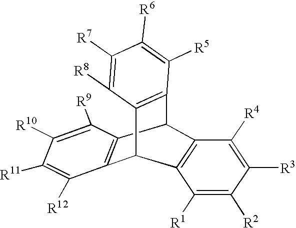

where R¹˜R¹² can be identical or different and R¹˜R¹² are independently selected from the group consisting of the following: aryl group having one or more substituents; heterocyclic aryl group having one or more substituents; non-aryl group having one or more substituents;

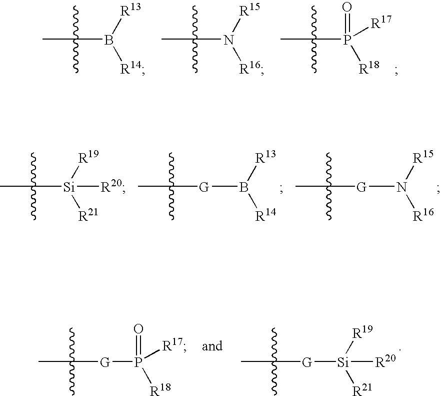

The substituent is selected from the group consisting of the following: H atom, halogen atom (such as F, Cl, Br, I); aryl group, halogen substituted aryl group, halogen substituted aralkyl group, haloalkyl substituted aryl group, haloalkyl substituted aralkyl group, aryl substituted C1-C20 alkyl group; electron donating group such as C1-C20 alkyl group, C1-C20 cycloalkyl group (such as methyl, ethyl, butyl, cyclohexyl), C1-C20 alkoxy group, C1-C20 substituted amino group, substituted aromatic amino group (such as aniline); or electron withdrawing group [such as halogen atom, nitrile group, nitro group, carbonyl group, cyano (—CN) group] and halogen substituted C1-C20 alkyl group (such as CF₃); and heterocyclic group.

The above G is selected from the group consisting of the following: aryl group having one or more substituents; heterocyclic aryl group having one or more substituents; heterocyclic group having one or more substituents. The substituent of G is selected from the group consisting of the following: H atom, halogen atom (such as F, Cl, Br, I); aryl group, halogen substituted aryl group, halogen substituted aralkyl group, haloalkyl substituted aryl group, haloalkyl substituted aralkyl group, aryl substituted C1-C20 alkyl group; electron donating group such as C1-C20 alkyl group, C1-C20 cycloalkyl group (such as methyl, ethyl, butyl, cyclohexyl), C1-C20 alkoxy group, C1-C20 substituted amino group, substituted aromatic amino group (such as aniline); or electron withdrawing group [such as halogen atom, nitrile group, nitro group, carbonyl group, cyano (—CN) group] and halogen substituted C1-C20 alkyl group (such as CF₃); and heterocyclic group.

The above R¹³˜R²¹ can be identical or different and R¹³˜R²¹ are independently selected from the group consisting of the following: H atom; C1-C20 alkyl group, C1-C20 cycloalkyl group (such as methyl, ethyl, butyl, cyclohexyl); C1-C20 alkoxy group; amino group aryl group having one or more substituents; and heterocyclic aryl group having one or more substituents. The substituent of R¹³˜R²¹ is independently selected from the group consisting of the following: H atom, halogen atom, aryl group, halogen substituted aryl group, halogen substituted aralkyl group, haloalkyl substituted aryl group, haloalkyl substituted aralkyl group, aryl substituted C1-C20 alkyl group, C1-C20 alkyl group, C1-C20 cycloalkyl group, C1-C20 alkoxy group, C1-C20 substituted amino group, substituted aromatic amino group, electron withdrawing group substituted C1-C20 alkyl group, halogen substituted C1-C20 alkyl group (such as CF₃), heterocyclic group, nitrile group, nitro group, carbonyl group, and cyano group (—CN). According to this embodiment, in the structure of the triptycene derivative, R¹˜R¹² are not H atoms simultaneously.

According to this embodiment, the aryl group is selected from the group consisting of the following: phenyl, naphthyl, diphenyl, anthryl, pyrenyl, phenanthryl, fluorene, or other multi-phenyl group.

The heterocyclic aryl group is selected from the group consisting of the following: pyrane, pyrroline, furan, benzofuran, thiophene, benzothiophene, pyridine, quinoline, isoquinoline, pyrazine, pyrimidine, pyrrole, pyrazole, imidazole, indole, thiazole, isothiazole, oxazole, isoxazole, benzothiazole, benzoxazole, 1,2,4-triazole, 1,2,3-triazole, 1,2,3,4-tetraazole, phenanthroline, or other heterocyclic aryl group.

The non-aryl group is selected from the group consisting of the following or any combination thereof: H atom, halogen atom; C1-C20 alkyl group, C1-C20 cycloalkyl group(such as methyl, ethyl, butyl, cyclohexyl); C1-C20 alkoxy group; amino group; nitrile group; nitro group, carbonyl group; cyano group (-CN); C1-C20 aryl substituted haloalkyl group; C1-C20 aralkyl substituted haloalkyl group; aryl substituted C1-C20 alkyl group, aryl substituted amino group, and C1-C20 alkyl substituted amino group.

### General Process for Fabricating an Organic Light Emitting Device

An ITO glass with etched circuitry is placed in a cleaning liquid (neutral cleanser: deionized water=1:50) and carried out supersonic oscillation for 5 minutes. Then, the ITO glass is brushed by a soft brush and sequentially carried out the following steps: placing in 50 mL of deionized water, oscillating in electronic grade acetone for 5 minutes, and drying by nitrogen. The cleaned ITO glass is placed in an ultraviolet-ozone machine for 5 minutes. Finally, the ITO glass with the ITO surface facing downward is provided on the substrate holder in an evaporator. The chamber is vacuumed. The process of evaporating thin film does not start until the pressure in the chamber reaches 5×10⁻⁶ torr. The conditions of evaporation are as follows. The evaporation rate for the organic films is controlled at 1˜2 Å/s and then the expected organic films are evaporated sequentially. The evaporation rate of magnesium for the metal film is 5 Å/s while that of silver is 0.5 Å/s (Mg:Ag=10:1). The Mg—Ag co-evaporated metal film has a thickness of 55 nm. Finally, a silver layer having a thickness of 100 nm as a protection layer is formed. In the case of choosing LiF/Al system as metal, firstly LiF is evaporated with a rate of 0.1 A/ s to form a film with a thickness of 1 nm and secondly an aluminum layer having a thickness of 100 nm as a protection layer is formed. During the process of evaporation, the rotational speed of the device is about 20 rpm. After the evaporation process is finished, the metal electrode is stayed for 20 minutes to cool and then the chamber is filled with nitrogen until the pressure returns normal pressure.

On the other hand, after the OLED device is fabricated, the EL spectrum and CIE corrdination of the device are measured by F-4500 Hitachi. In addition, the properties, such as current, voltage, and brightness of the device are measured by Kiethley 2400 programmable voltage-current source. The measurements are carried out at room temperature (about 25° C.) and 1 atm.

### EXAMPLE 12

By the general process of fabricating OLED, TCTP is the host emitting material and doped with blue phosphorescence materials to form OLEDs. The doped blue phosphorescence materials have the following structures:

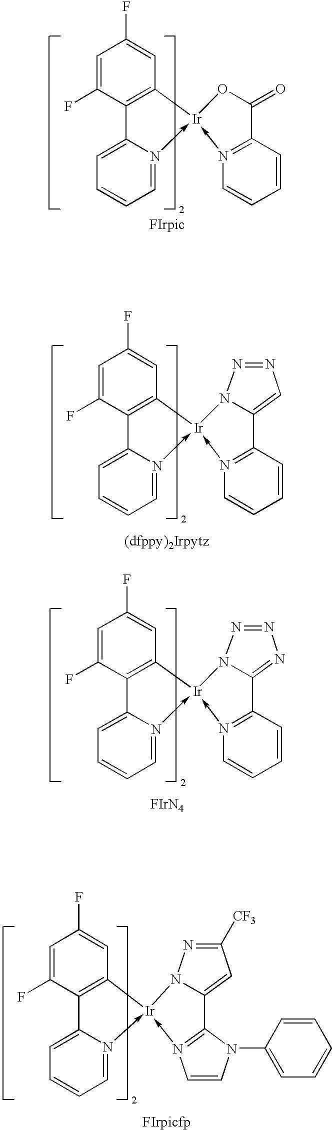

The structure of each device is shown in the following:
Device 3A: NPB(30)/TCTP:FIrpic(7%)(30)/BCP(15)/Alq(30)
Device 3B: NPB(30)/mcp(20)/TCTP:FIrpic(7%)(30)/BCP(15)/Alq(30)
Device 3C: TCTA(30)/mcp(20)/TCTP:FIrpic(7%)(30)/BCP(15)/Alq(30)
Device 3D: NPB(30)/mcp(20)/TCTP:FIrpic(6.3%)(30)/TPBI(30)

The cathode of the devices 3A˜3D is Li (1)/Al(100). The thickness of the devices are repsented in nm. The optical properties and efficiency of the devices 3A˜3D are measured and shown in Table 3-1.

TABLE 3-1

| device | V_{d}^{a} | Lum^{b} | ηₑₓₜ^{c} | η_{c}^{d} | ηₚ^{e} | |
|---|---|---|---|---|---|---|
| (%) | (V) | (cd/m²) | (%) | cd/A | (lm/W) | C.I.E |
| 3A | 4.6 | 37992@14.5 V | 5.8@10.0 V | 12.2@10.0 V | 4.1@9.0 V | 0.14, 0.34@8 v |
| 3B | 4.6 | 46739@16.0 V | 10.1@6.5 V | 21.9@6.5 V | 12.4@5.0 V | 0.14, 0.36@8 v |
| 3C | 5.6 | 37385@18.0 V | 6.4@11.5 V | 13.8@11.5 V | 4.0@10.0 V | 0.14, 0.35@8 v |
| 3D | 5.1 | 17692@13.0 V | 4.9@9.0 V | 8.8@9.0 V | 3.2@8.0 V | 0.13, 0.28@8 v |

^{a}Drive voltage (V_{d});
^{b}maximum luminescence (L);
^{c}maximum external quantum efficiency (ηₑₓₜ);
^{d}maximum current efficiency (η_{c});
^{e}maximum power efficiency (ηₚ).

Device 3E:NPB(30)/mcp(20)/TCTP:(dfppy)₂Ir(pytz)(7%)(30)/TPBI(30)
Device 3F:NPB(30)/mcp(20)/TCTP:FIrN₄(7%)(30)/BCP(15)/Alq(30)
Device 3G:NPB(25)/mcp(25)/TCTP:FIrpicfp(7.7%)(30)/BCP(15)/Alq(30)

The cathode of the devices 3E˜3G is Li (1)/Al(100). The thichness of the devices are repsented in nm. The optical properties and efficiency of the devices 3E˜3G are measured and shown in Table 3-2.

TABLE 3-2

| | V_{d}^{a} | Lum^{b} | ηₑₓₜ^{c} | η_{c}^{d} | ηₚ^{e} | |
|---|---|---|---|---|---|---|
| device (%) | (V) | (cd/m²) | (%) | cd/A | (lm/W) | C.I.E |
| 3E | 5.0 | 16777@13.5 V | 7.9@8.0 V | 12.2@8.0 V | 5.2@7.0 V | 0.14, 0.22@8 v |
| 3F | 5.4 | 7777@16.0 V | 4.5@6.5 V | 9.0@10.5 V | 3.0@8.5 V | 0.14, 0.29@8 v |
| 3G | 7.0 | 15462@17.5 V | 4.1@12.0 V | 8.4@12.0 V | 2.5@9.5 V | 0.14, 0.31@8 v |

^{a}Drive voltage (V_{d});
^{b}maximum luminescence (L);
^{c}maximum external quantum efficiency (ηₑₓₜ);
^{d}maximum current efficiency (η_{c}); e. maximum power efficiency (ηₚ).

### EXAMPLE 13

By the general process of fabricating OLED, TPTP is the host emitting material and doped with blue phosphorescence materials to form OLEDs. The doped blue phosphorescence materials have the following structures:

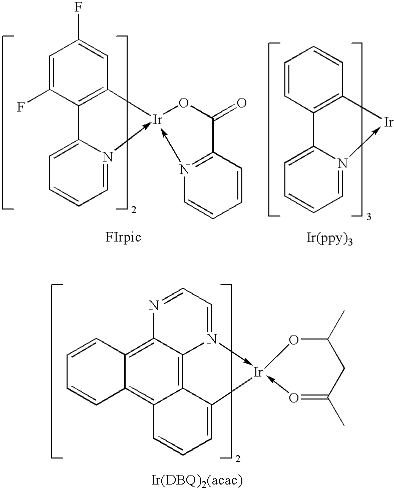

The structure of each device is shown in the following:
Device 3H: NPB(40)/TPTP:FIrpic(7%)(30)/BCP(15)/Alq(30)
Device 3I: NPB(30)/mcp(20)/TPTP:FIrpic(6.7%)(30)/BCP(15)/Alq(30)
Device 3J: TCTA(30)/mcp(20)/TPTP:FIrpic(6.7%)(30)/BCP(15)/Alq(30)
Device 3K: NPB(30)/mcp(20)/TPTP:FIrpic(6.7%)(30)/TPBI(30)

The cathode of the devices 3H˜3K is Li (1)/Al(100). The thichness of the devices are repsented in nm. The optical properties and efficiency of the devices 3H˜3K are measured and shown in Table 3-3.

TABLE 3-3

| | V_{d}^{a} | Lum^{b} | ηₑₓₜ^{c} | η_{c}^{d} | ηₚ^{e} | |
|---|---|---|---|---|---|---|
| device (%) | (V) | (cd/m²) | (%) | cd/A | (lm/W) | C.I.E |
| 3H | 3.1 | 5625@11.0 V | 0.6@6.0 V | 1.2@4.5 V | 1.2@3.0 V | 0.14, 0.30@8 v |
| 3I | 4.7 | 32313@14.0 V | 7.8@7.0 V | 16.7@7.0 V | 8.2@6.0 V | 0.14, 0.35@8 v |
| 3J | 6.6 | 28618@19.5 V | 8.1@7.5 V | 17.3@7.5 V | 7.2@7.5 V | 0.14, 0.35@8 v |
| 3K | 4.7 | 17266@13.0 V | 7.6@7.0 V | 14.0@7.0 V | 6.8@6.0 V | 0.13, 0.29@8 v |

^{a}Drive voltage (V_{d});
^{b}maximum luminescence (L);
^{c}maximum external quantum efficiency (ηₑₓₜ);
^{d}maximum current efficiency (η_{c});
^{e}maximum power efficiency (ηₚ).

TPTP is the host emitting material and doped with the green phosphorescence material Ir(ppy)3 to form OLEDs. The structures of devices are shown in the following:
Device 3L: NPB(30)/mcp(20)/TPTP: Ir(ppy)₃(7.3%)(30)/BCP(15)/Alq(30)
Device 3M: TCTA(30)/mcp(20)/TPTP:Ir(ppy)₃(6.8%)(30)/BCP(15)/Alq(30)

The cathode of the devices 3L and 3M is Li (1)/Al(100). The thichness of the devices are repsented in nm. The optical properties and efficiency of the devices 3L and 3M are measured and shown in Table 3-4.

TABLE 3-4

| Device | V_{d}^{a} | Lum^{b} | ηₑₓₜ^{c} | η_{c}^{d} | ηₚ^{e} | |
|---|---|---|---|---|---|---|
| (%) | (V) | (cd/m²) | (%) | cd/A | (lm/W) | C.I.E |
| 3L | 4.2 | 58930@16.0 V | 7.2@11.0 V | 26.1@11.0 V | 7.9@10.0 V | 0.24, 0.64@8 v |
| 3M | 4.3 | 41054@13.5 V | 11.5@8.0 V | 41.2@8.0 V | 19.5@6.0 V | 0.23, 0.66@8 v |

^{a}Drive voltage (V_{d});
^{b}maximum luminescence (L);
^{c}maximum external quantum efficiency (ηₑₓₜ);
^{d}maximum current efficiency (η_{c});
^{e}maximum power efficiency (ηₚ).

TPTP is the host emitting material and doped with the red phosphorescence material Ir(DBQ)₂(acac) to form OLEDs. The structures of devices are shown in the following:
Device 3N: TCTA(30)/mcp(20)/TPTP:Ir(DBQ)₂(acac)(7%)(30)/BCP(10)/Alq(40)
Device 3O: TCTA(30)/mcp(20)/TPTP:Ir(DBQ)₂(acac)(10%)(30)/BCP(15)/Alq(30)
Device 3P: NPB(30)/mcp(20)/TPTP:Ir(DBQ)₂(acac)(10%)(30)/BCP(15)/Alq(30)
Device 3Q: NPB(30)/mcp(20)/TPTP:Ir-red(10%)(30)/BCP(15)/Alq(30)

The cathode of the devices 3N˜3Q is Li (1)/Al(100). The thichness of the devices are repsented in nm. The optical properties and efficiency of the devices 3N˜3Q are measured and shown in Table 3-5.

TABLE 3-5

| Device | V_{d}^{a} | Lum^{b} | ηₑₓₜ^{c} | η_{c}^{d} | ηₚ^{e} | |
|---|---|---|---|---|---|---|
| (%) | (V) | (cd/m²) | (%) | cd/A | (lm/W) | C.I.E |
| 3N | 3.9 | 48244@17.0 V | 3.8@10.5 V | 8.3@10.5 V | 2.6@9.5 V | 0.57, 0.39@8 v |
| 3O | 4.1 | 37048@14.0 V | 3.6@10.0 V | 7.1@10.0 V | 2.4@9.0 V | 0.61, 0.38@8 v |
| 3P | 4.3 | 51591@15.5 V | 9.8@6.5 V | 19.0@6.5 V | 9.4@5.5 V | 0.62, 0.38@8 v |
| 3Q | 4.7 | 16969@16.5 V | 7.6@8.0 V | 10.8@8.0 V | 4.7@7.0 V | 0.65, 0.33@8 v |

^{a}Drive voltage (V_{d});
^{b}maximum luminescence (L);
^{c}maximum external quantum efficiency (ηₑₓₜ);
^{d}maximum current efficiency (η_{c});
^{e}maximum power efficiency (ηₚ).

### EXAMPLE 14

By the general process of fabricating OLED, TBP, TPA, TPSi, and TPC are the host emitting materials and doped with guest phosphorescence materials to form OLEDs. The structure of each device is shown in the following:
Device TBP1:NPB(30)/TCTA(20)/TBP:FIrpic(6%)(30)/BCP(10)/Alq(30)
Device TPA6:NPB(30)/mCP(20)/TPA:FIrpic(6%)(30)/BCP(10)/Alq(30)
Device TPA8:NPB(30)/mCP(20)/TPA: Ir(ppy)₃(6%)(30)/TPBI(30)
Device TPSi3:NPB(30)/TCTA(20)/TPSi:FIrpic(6%)(30)/BCP(10)/Alq(30)
Device TPC2:TPD(30)/mCP(20)/TPC:FIrpic(6%)(30)/BCP(15)/Alq(30)

The cathode of the devices is Li (1)/Al(100). The thichness of the devices are repsented in nm. The optical properties and efficiency of the devices are measured and shown in Table 3-6.

TABLE 3-6

| | V_{d}^{a} | Lum^{b} | ηₑₓₜ^{c} | η_{c}^{d} | ηₚ^{e} | |
|---|---|---|---|---|---|---|
| device | (V) | (cd/m²) | (%) | (cd/A) | (lm/W) | C.I.E.^{f} |
| TBP1 | 6.8 | 7235 | 2.4@11.5 V | 5.3@11.5 V | 1.7@9 V | (0.14, 0.34) |
| TPA6 | 4.6 | 13473 | 4.4@6.5 V | 9.7@6.5 V | 5@5.5 V | (0.14, 0.35) |
| TPA8 | 4.7 | 31533 | 8.8@7 V | 31.5@7 V | 18.7@5 V | (0.22, 0.64) |
| TPSi3 | 4.5 | 8442 | 5.0@5 V | 10.5@5 V | 6.6@5 V | (0.15, 0.33) |
| TPC2 | 4.5 | 13729 | 3.8@6.5 V | 7.8@6.5 V | 4.3@5 V | (0.14, 0.32) |

^{a}Drive voltage (V_{d}),
^{b}Luminescence (Lum),
^{c}Maximum external quantum efficency (ηₑₓₜ),
^{d}Maximum current efficiency (η_{c}),
^{e}Maximum power efficiency (ηₚ),
^{f}C.I.E_{x,y} at 8 V.

In this embodiment, the triptycene derivative is applied as a host material, a single-layer emitting material, an electron transport material, or a hole transport material in an organic electroluminescence device. On the other hand, the triptycene derivative has the characteristics of electron and hole transport to be applied as an electron transport material or a hole transport material in other electronic devices, besides in an organic electroluminescence device.

According to the invention, the triptycene derivative the excellent heat stability to make the lifetime of the organic electronic device effectively increased. In addition, the triptycene derivative has high triplet-state energy difference, which can not be provided by various common blue, green, red phosphorescent host materials, and can be used together with various common phosphorescent materials, such as the iridium (Ir), platinum (Pt), and osmium (Os) metal complexes. Therefore, this present invention does have the economic advantages for industrial applications.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A triptycene derivative, comprising: the following general structure: 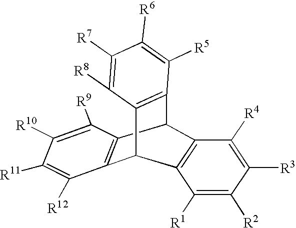
where R¹˜R¹² can be identical or different and R¹˜R¹² are independently selected from the group consisting of the following: aryl group having one or more substituents; heterocyclic aryl group having one or more substituents; non-aryl group having one or more substituents; 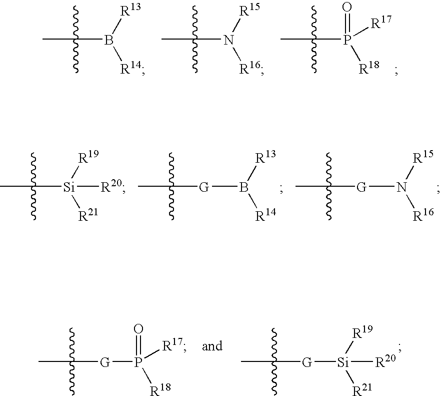
wherein G is selected from the group consisting of the following: aryl group having one or more substituents; heterocyclic aryl group having one or more substituents; heterocyclic group having one or more substituents; said substituent is independently selected from the group consisting of the following: H atom, halogen atom, aryl group, halogen substituted aryl group, halogen substituted aralkyl group, haloalkyl substituted aryl group, haloalkyl substituted aralkyl group, aryl substituted C1-C20 alkyl group, C1-C20 alkyl group, C1-C20 cycloalkyl group, C1-C20 alkoxy group, substituted C1-C20 amino group, substituted aromatic amino group, electron withdrawing group substituted C1-C20 alkyl group, and heterocyclic group;
R¹³˜R²¹ can be identical or different and R¹³˜R²¹ are independently selected from the group consisting of the following: H atom, C1-C20 alkyl group, C1-C20 cycloalkyl group, C1-C20 alkoxy group, amino group, aryl group having one or more substituents, and heterocyclic aryl group having one or more substituents; said substituent of R¹³˜R²¹ is independently selected from the group consisting of the following: H atom, halogen atom, aryl group, halogen substituted aryl group, halogen substituted aralkyl group, haloalkyl substituted aryl group, haloalkyl substituted aralkyl group, aryl substituted C1-C20 alkyl group, C1-C20 alkyl group, C1-C20 cycloalkyl group, C1-C20 alkoxy group, C1-C20 substituted amino group, substituted aromatic amino group, electron withdrawing group substituted C1-C20 alkyl group, halogen substituted C1-C20 alkyl group (such as CF₃), heterocyclic group, nitrile group, nitro group, carbonyl group, and cyano group (—CN); R¹˜R¹² are not H atoms simultaneously.

2. The derivative according to claim 1, wherein said aryl group comprises one functional group selected from the group consisting of the following: phenyl, naphthyl, diphenyl, anthryl, pyrenyl, phenanthryl, fluorene, or other multi-phenyl group.

3. The derivative according to claim 1, wherein said heterocyclic aryl group comprises one functional group selected from the group consisting of the following: pyrane, pyrroline, furan, benzofuran, thiophene, benzothiophene, pyridine, quinoline, isoquinoline, pyrazine, pyrimidine, pyrrole, pyrazole, imidazole, indole, thiazole, isothiazole, oxazole, isoxazole, benzothiazole, benzoxazole, 1,2,4-triazole, 1,2,3-triazole, 1,2,3,4-tetraazole, phenanthroline, or other heterocyclic aryl group.

4. The derivative according to claim 1, wherein said non-aryl group is selected from the group consisting of the following or any combination thereof: H atom, halogen atom, C1-C20 alkyl group, C1-C20 cycloalkyl group, C1-C20 alkoxy group, amino group, nitrile group, nitro group, carbonyl group, cyano group (—CN), halogen substituted C1-C20 alkyl group, aryl substituted C1-C20 alkyl group, aryl substituted amino group, and C1-C20 alkyl substituted amino group.

5. The derivative according to claim 1, wherein the derivative is utilized in an organic electroluminescence and/or phosphorescence device.

6. The derivative according to claim 1, wherein the derivative is utilized as a host material or a single-layer emitter in an organic electroluminescence and/or phosphorescence device.

7. The derivative according to claim 1, wherein the derivative is utilized as an electron transport material in an organic electronic device.

8. The derivative according to claim 1, wherein the derivative is utilized as a hole transport material in an organic electronic device.

9. An organic light emitting device, comprising:
a pair of electrodes; and
at least one organic layer provided between said electrodes;
wherein said at least one organic layer comprises one emitter layer and at least one of said organic layers comprises a triptycene derivative, having the following general structure: 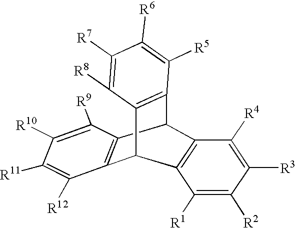
where R¹˜R¹² can be identical or different and R¹˜R¹² are independently selected from the group consisting of the following: aryl group having one or more substituents; heterocyclic aryl group having one or more substituents; non-aryl group having one or more substituents; 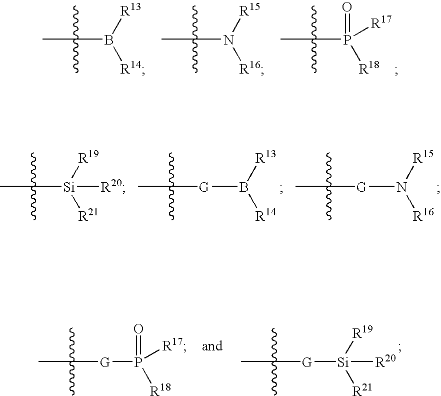
wherein G is selected from the group consisting of the following: aryl group having one or more substituents; heterocyclic aryl group having one or more substituents; heterocyclic group having one or more substituents; said substituent is independently selected from the group consisting of the following: H atom, halogen atom, aryl group, halogen substituted aryl group, halogen substituted aralkyl group, haloalkyl substituted aryl group, haloalkyl substituted aralkyl group, aryl substituted C1-C20 alkyl group, C1-C20 alkyl group, C1-C20 cycloalkyl group, C1-C20 alkoxy group, substituted C1-C20 amino group, substituted aromatic amino group, electron withdrawing group substituted C1-C20 alkyl group, and heterocyclic group;
R¹³˜R²¹ can be identical or different and R¹³˜R²¹ are independently selected from the group consisting of the following: H atom, C1-C20 alkyl group, C1-C20 cycloalkyl group, C1-C20 alkoxy group, amino group, aryl group having one or more substituents, and heterocyclic aryl group having one or more substituents; said substituent of R¹³˜R²¹ is independently selected from the group consisting of the following: H atom, halogen atom, aryl group, halogen substituted aryl group, halogen substituted aralkyl group, haloalkyl substituted aryl group, haloalkyl substituted aralkyl group, aryl substituted C1-C20 alkyl group, C1-C20 alkyl group, C1-C20 cycloalkyl group, C1-C20 alkoxy group, C1-C20 substituted amino group, substituted aromatic amino group, electron withdrawing group substituted C1-C20 alkyl group, halogen substituted C1-C20 alkyl group (such as CF₃), heterocyclic group, nitrile group, nitro group, carbonyl group, and cyano group (—CN); R¹˜R¹² are not H atoms simultaneously.

10. The device according to claim 9, wherein said aryl group comprises one functional group selected from the group consisting of the following: phenyl, naphthyl, diphenyl, anthryl, pyrenyl, phenanthryl, fluorene, or other multi-phenyl group.

11. The device according to claim 9, wherein said heterocyclic aryl group comprises one functional group selected from the group consisting of the following: pyrane, pyrroline, furan, benzofuran, thiophene, benzothiophene, pyridine, quinoline, isoquinoline, pyrazine, pyrimidine, pyrrole, pyrazole, imidazole, indole, thiazole, isothiazole, oxazole, isoxazole, benzothiazole, benzoxazole, 1,2,4-triazole, 1,2,3-triazole, 1,2,3,4-tetraazole, phenanthroline, or other heterocyclic aryl group.

12. The device according to claim 9, wherein said non-aryl group is selected from the group consisting of the following or any combination thereof: H atom, halogen atom, C1-C20 alkyl group, C1-C20 cycloalkyl group, C1-C20 alkoxy group, amino group, nitrile group, nitro group, carbonyl group, cyano group (—CN), halogen substituted C1-C20 alkyl group, aryl substituted C1-C20 alkyl group, aryl substituted amino group, and C1-C20 alkyl substituted amino group.

13. The device according to claim 9, wherein the triptycene derivative is utilized in the emitter layer of the organic light emitting device.

14. The device according to claim 9, wherein the triptycene derivative is utilized as a host material in the emitter layer of the organic light emitting device.

15. The device according to claim 9, wherein the triptycene derivative is utilized in an electron transport material of the organic light emitting device.

16. The device according to claim 9, wherein the triptycene derivative is utilized in a hole transport material of the organic light emitting device.

17. The device according to claim 16, wherein said emitter layer further comprises a guest emitting material and said guest emitting material comprises a transition metal complex.

18. The device according to claim 17, wherein the transition metal of said transition metal complex is selected from the group consisting of the following: iridium (Ir), platinum (Pt), and osmium (Os).

19. The device according to claim 17, wherein said guest emitting material is blue phosphorescent.

20. The device according to claim 17, wherein said guest emitting material is red phosphorescent.

21. The device according to claim 17, wherein said guest emitting material is green phosphorescent.

22. The device according to claim 9, wherein said at least one organic layer comprises one compound having the structure selected from the group consisting the following: 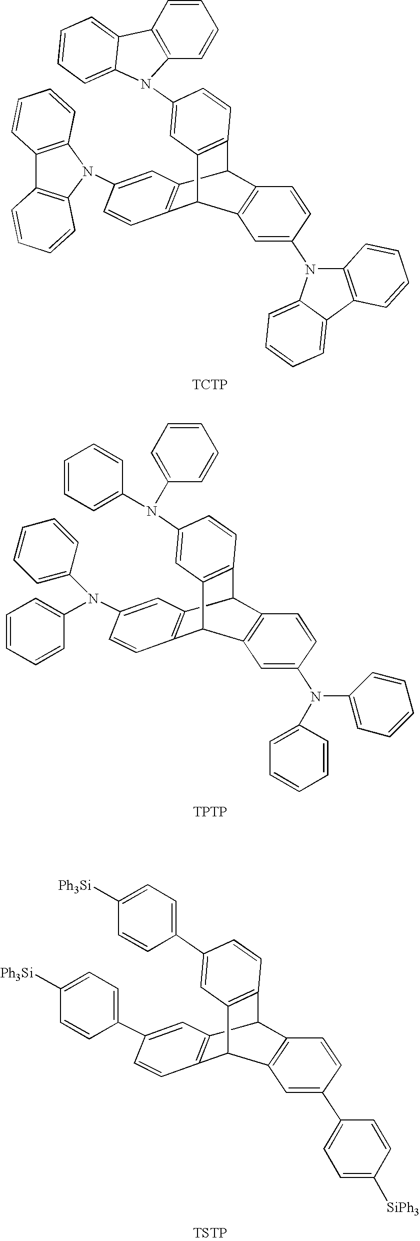 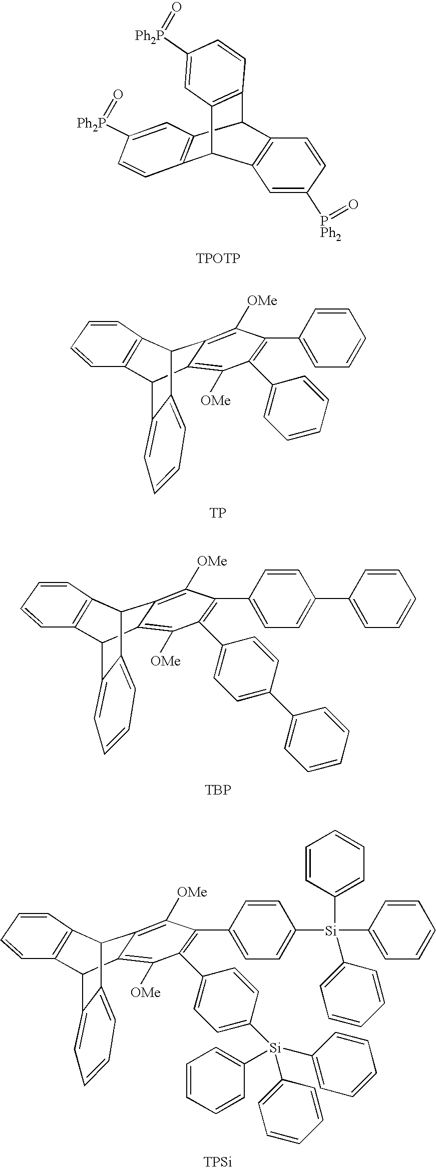 [chemical structure]
